(19) United States
(12) Patent Application Publication
(10) Pub. No.: US 2024/0255511 A1
(43) Pub. Date: Aug. 1, 2024

(54) DEVICE FOR DIAGNOSING LIVER CANCER

(71) Applicant: HUMAN METABOLOMICS INSTITUTE, INC., SHENZHEN, GUANGDONG (CN)

(72) Inventors: Wei Jia, Shenzhen, Guangdong (CN); Guoxiang Xie, Shenzhen, Guangdong (CN); Tianlu Chen, Shenzhen, Guangdong (CN)

(21) Appl. No.: 18/564,640

(22) PCT Filed: May 27, 2022
(86) PCT No.: PCT/CN2022/095737

§ 371 Date: Nov. 28, 2023

(30) Foreign Application Priority Data
May 28, 2021 (CN) 202110593051.X

(51) Int. Cl. G01N 33/574; G01N 30/02; G01N 30/72; G01N 30/88

(52) U.S. Cl. CPC G01N 33/57438; G01N 30/7233; G01N 30/88; G01N 2030/027; G01N 2030/8822; G01N 2570/00

(57) ABSTRACT

Provided in the present invention is a device for diagnosing liver cancer. The device determines the level of a biomarker in a biological sample of a subject to be a diagnostic indicator, wherein the level of the biomarker is selected from the level of one or more of taurocholic acid, taurochenodeoxycholic acid, glycocholic acid, glycochenodeoxycholic acid, linolelaidic acid (C18:2n6t), maltotriose, maltose and/or lactose, α-linolenic acid, β-alanine, sebacic acid, 2-methylvaleric acid, valeric acid, isovaleric acid and hexanoic acid, and/or the ratio of secondary bile acid to primary bile acid, and/or the ratio of glycine-conjugated primary bile acid to taurine-conjugated primary bile acid, the primary bile acid being selected from cholic acid and chenodeoxycholic acid, and the secondary bile acid comprising deoxycholic acid, lithocholic acid and ursodeoxycholic acid. The diagnosis device of the present invention can be used for early diagnosis of liver cancer and liver cirrhosis, thereby buying time for patients and improving the clinical treatment effect.

The present application claims the priority of a Chinese patent application No. 202110593051X filed on May 28, 2021. The entire contents of the Chinese patent application are incorporated into the present application by reference.

### FIELD OF TECHNOLOGY

The present invention belongs to the field of biology and specifically relates to a diagnostic device for hepatocellular carcinoma. The device is used with levels of biomarkers in a biological sample of a subject as detection indicators. The present invention also relates to a set of biomarkers that can be used in diagnosis of hepatocellular carcinoma.

### BACKGROUND

Hepatocellular carcinoma (HCC) is a primary malignant tumor of the liver, which is mainly developed in patients with chronic liver disease and liver cirrhosis. At present, the HCC is the third cause of death from cancer in the global world. The HCC has highest morbidity in Asia and Africa, and the chronic liver disease is likely to be developed and then progressed into the HCC due to high epidemicity of hepatitis B and hepatitis C in these regions. In the past, patients with HCC were usually confirmed when having advanced symptoms of pain in the upper right quadrant, weight loss and liver decompensation. At present, puncture biopsy is commonly used as a gold standard for diagnosis of the hepatocellular carcinoma in clinical practice. However, the method has great limitations, such as invasive tests, sampling errors, operation and read errors of pathologists and the like. Other methods for predicting the hepatocellular carcinoma include measuring the content of alpha-fetoprotein (AFP) in serum during routine screening to improve the early diagnosis rate of the HCC. However, the technology also has obvious limitations, such as low sensitivity and poor specificity. It is expected that threats caused by the HCC will be continuously increased in the next few years. Therefore, it is necessary to explore other biological markers as indicators for early screening of the hepatocellular carcinoma or for diagnosis in combination with a variety of biological markers, so as to improve the sensitivity and specificity in diagnosis of the early hepatocellular carcinoma and relieve the pain of patients during puncture.

### SUMMARY

The present invention provides a diagnostic device for hepatocellular carcinoma. The diagnostic device is used with levels of biomarkers in a biological sample of a subject as detection indicators, and can be used for realizing a variety of purposes including risk assessment, screening and diagnosis related to hepatocellular carcinoma and liver cirrhosis. The diagnostic device for hepatocellular carcinoma of the present invention can be used in various product forms. The present invention further provides a use method of the diagnostic device for hepatocellular carcinoma. The present invention further provides a set of biomarkers with predictive and diagnostic capabilities for hepatocellular carcinoma and liver cirrhosis and use thereof.

All terms and abbreviations used in the present invention are described below.

The term “diagnosis” used in the present invention is used for facilitating the expression of purposes, but is not understood as being limited to “diagnosis” behaviors defined in accordance with clinical standards. The “diagnosis” of the present invention includes the “diagnosis” behaviors defined in accordance with clinical standards, and also includes all processes and behaviors that lead to valuable conclusions by evaluating diagnostic indicators provided by the present invention, including but not limited to the following purposes and use methods: assessing the risk level of hepatocellular carcinoma or liver cirrhosis in a subject, for example, use in general screening in physical examination; regularly monitoring a high-risk population; evaluating the efficacy of drugs for treatment of liver cirrhosis or hepatocellular carcinoma or drugs for potential treatment of liver cirrhosis or hepatocellular carcinoma; evaluating substances or treatment means that may lead to the risk of liver cirrhosis or hepatocellular carcinoma and the like. All the listed exemplary purposes are included in the scope defined by the “diagnosis” of the present invention.

Accordingly, the diagnostic device of the present invention may be used for purposes including but not limited to early assessment of hepatocellular carcinoma or liver cirrhosis in a subject, general screening in physical examination, clinical diagnosis and drug evaluation, and can be used separately to obtain corresponding conclusions or used in combination with other detection devices or detection indicators (such as alpha-fetoprotein) for diagnosis. Embodiments show that the accuracy and the reliability in clinical diagnosis can be improved by using the diagnostic device of the present invention in combination with the alpha-fetoprotein.

The diagnostic indicators of the present invention include ratios obtained by calculation based on original data, which are expressed as “/” in the specification. For example, taurochenodeoxycholic acid/glycochenodeoxycholic acid represents the ratio of taurochenodeoxycholic acid to glycochenodeoxycholic acid, which is the ratio of the two substances based on a same sample, a same detection method and a same unit of value as understood by persons of ordinary skill in the art.

Unless defined in other parts of the specification, all technical terms and scientific terms used in the specification have the same meanings as generally understood by persons of ordinary skill in the art to which the present invention belongs. As used in the specification and the attached claims, the singular forms “one” and “the” include one or more objects referred to, unless different meanings are obviously indicated in the content. For example, the term “component” referred to includes a combination of one or more of components and the like.

Unless defined in other parts of the specification, abbreviations used in the present invention have the following meanings:
HCC: hepatocellular carcinoma,
CLD: chronic liver disease,
HBV: hepatitis B virus,
CA: cholic acid,
TCA: taurocholic acid,
TCDCA: taurochenodeoxycholic acid,
GCA: glycocholic acid,
DCA: deoxycholic acid,
LCA: lithocholic acid,
CDCA: chenodeoxycholic acid,
UDCA: ursodeoxycholic acid, and
GCDCA: glycochenodeoxycholic acid.

The present invention is realized by adopting the following technical schemes.

In a first aspect, the present invention provides a diagnostic device for hepatocellular carcinoma. The device is used by determining levels of biomarkers in a biological sample of a subject as diagnostic indicators. The levels of biomarkers are selected from levels of one or more of taurocholic acid, taurochenodeoxycholic acid, glycocholic acid, glycochenodeoxycholic acid, trans-linoleic acid (C18:2n6t), maltotriose, maltose and/or lactose, α-linolenic acid, β-alanine, sebacic acid, 2-methylvaleric acid, valeric acid, isovaleric acid and caproic acid, and/or the ratio of a secondary bile acid to a primary bile acid, and/or the ratio of a combination of glycine and a primary bile acid to a combination of taurine and a primary bile acid. The primary bile acid is selected from cholic acid and chenodeoxycholic acid. The secondary bile acid includes deoxycholic acid, lithocholic acid and ursodeoxycholic acid.

The above diagnostic indicators may be used separately or in combination. For example, as a specific embodiment, the levels of biomarkers are selected from levels of one or more of taurocholic acid, taurochenodeoxycholic acid, glycocholic acid, glycochenodeoxycholic acid and trans-linoleic acid (C18:2n6t). As a specific embodiment, the ratio of a secondary bile acid to a primary bile acid may be selected from deoxycholic acid/cholic acid, lithocholic acid/chenodeoxycholic acid and ursodeoxycholic acid/chenodeoxycholic acid. As a specific embodiment, the ratio of a combination of glycine and a primary bile acid to a combination of taurine and a primary bile acid may be selected from taurochenodeoxycholic acid/glycochenodeoxycholic acid.

The diagnostic indicators of the present invention may further include alpha-fetoprotein. It is proved in embodiments of the present invention that when the diagnostic indicators of the present invention are used in combination with the alpha-fetoprotein, a better diagnostic effect is achieved during prediction and distinguishing of healthy persons, chronic liver disease, liver cirrhosis and hepatocellular carcinoma compared with separate use of the alpha-fetoprotein.

According to the diagnostic device for hepatocellular carcinoma of the present invention, a mammal, such as a human being, may be selected as the subject. The used biological sample may include a urine sample and a blood sample. When the blood sample is used, whole blood, plasma or serum of peripheral blood may be used. In the present invention, serum of peripheral blood of a subject is selected as a detection sample.

Determination of the levels of biomarkers is carried out for the purpose of quantitative detection and may include the following steps: treating the biological sample of the subject and then subjecting a biomarker combination in the biological sample to quantitative detection by a chromatography-mass spectrometry method in combination with a metabolomics analysis method, and the chromatography-mass spectrometry method in combination with the metabolomics analysis method includes a liquid chromatography-mass spectrometry method in combination with the metabolomics analysis method and a gas chromatography-mass spectrometry method in combination with the metabolomics analysis method.

The diagnostic device of the present invention may be used in various product forms. For exemplary purposes, the diagnostic device may be selected from a kit, a medical instrument, a computer system with a diagnostic module, and a detection device with a diagnostic module. The medical instrument, the kit and the like, as known by persons of ordinary skill in the art, are defined in accordance with provisions of relevant laws, regulations and policies of a local government, and have different classification methods and meanings in different countries and regions. The medical instrument, the kit and other terms of the present invention are used only in the form of describing diagnostic markers of the present invention, and do not have meanings defined in accordance with strict laws and regulations. In a case of being consistent with the purposes of the present invention, the medical instrument and the kit may be medical products registered by a relevant government department, or products or product combinations used by persons of ordinary skill in the art in a temporary use method and form.

As a specific embodiment, the diagnostic device of the present invention includes the following modules:
(1) a module for receiving a test sample of a test object;
(2) a module for detecting data of expression levels of diagnostic markers; and
(3) a module for generating a risk score based on the expression levels of biomarkers that are inputted as diagnostic indicators into a database, where the database contains a control expression profile associated with the test sample and a test method; the control expression profile is obtained in advance according to the test sample and the test method, and may be expressed as cutoff values of the tested diagnostic markers; and risk assessment is carried out by comparing the expression levels of the biomarkers in the test sample with preset cutoff values, and the test object is considered as having corresponding risk when having higher values than the cutoff values.

The diagnostic device of the present invention is illustrated below for exemplary purposes with a kit and a computer system as examples.

### (1) Kit

As a specific embodiment, the diagnostic device of the present invention may be used in the form of a kit, and the kit includes quantitative detection reagents for detecting diagnostic indicators. For exemplary purposes, for example, the quantitative detection reagents described in the embodiment further may include internal standards and biological sample extracting reagents, and further include software that can be used for counting and evaluating test results. The software may be set for operation in a computer.

### (2) Computer System

As a specific embodiment, the diagnostic device of the present invention may be a computer system with a diagnostic module and a detection device with a diagnostic module. The diagnostic module includes an information acquisition module and a hepatocellular carcinoma diagnosis module. The information acquisition module is at least used for acquiring information of diagnostic indicators. The hepatocellular carcinoma diagnostic module is at least used for performing the following operation: assessing whether a subject has hepatocellular carcinoma or liver cirrhosis based on the information of diagnostic indicators acquired by the information acquisition module.

In a second aspect, the present invention provides a biomarker combination for diagnosis of hepatocellular carcinoma. The biomarker combination includes one or more of taurocholic acid, taurochenodeoxycholic acid, glycocholic acid, glycochenodeoxycholic acid, trans-linoleic acid (C18:2n6t), maltotriose, maltose and/or lactose, α-linolenic acid, β-alanine, sebacic acid, 2-methylvaleric acid, valeric acid, isovaleric acid and caproic acid. These biomarkers or combinations thereof may be optionally used in combination with alpha-fetoprotein, thereby improving a diagnostic effect.

The present invention further provides a method for quantitative detection of the biomarkers that can be used for diagnosis of hepatocellular carcinoma.

The present invention has the following beneficial technical effects.

The present invention provides a detection device for hepatocellular carcinoma with specific detection indicators, which can be used for predicting and diagnosing patients with hepatocellular carcinoma and liver cirrhosis by detecting the detection indicators. The detection indicators adopted by the detection device of the present invention have an excellent distinguishing capability and can be used separately, or multiple indicators can be used in combination to improve the reliability of a detection effect. In addition, the liver cirrhosis and the hepatocellular carcinoma can be distinguished. When the detection indicators are used in combined with alpha-fetoprotein, a detection indicator commonly used for detecting the hepatocellular carcinoma in clinical practice, a diagnostic effect of the alpha-fetoprotein is obviously improved.

In order to make the purposes, technical schemes and effects of the present invention more clear and definite, the present invention is further explained in detail below in combination with the attached drawings and embodiments. It is to be understood that the specific embodiments described herein are intended only to explain the present invention, rather than to limit the present invention.

### BRIEF DESCRIPTION OF THE DRAWINGS

FIG. 1 shows diagnostic diagrams in Example 5.

FIG. 2 shows diagnostic diagrams in Example 6.

### DESCRIPTION OF THE EMBODIMENTS

The technical schemes of the present invention are described in detail below in combination with specific embodiments and attached drawings of the present invention. Obviously, the specific embodiments described herein are only a part of the embodiments for realizing the technical schemes of the present invention, and should not be understood as the entire embodiments. It is to be understood that the specific embodiments described herein are intended only to explain the present invention, rather than to limit the present invention. On the basis of the embodiments described herein, all other embodiments obtained by persons of ordinary skill in the art without making creative effort under inspiration shall fall within the scope of protection of the present invention.

A random forest model in the embodiments is selected by using LiveForest software of Shenzhen Human Metabolomics Institute, Inc., and the software has a copyright registration No. 2018SR227394 and a name: metabolomics-based machine learning diagnosis system for chronic liver disease V1.0.

### Example 1 Study of Biomarkers

A total of 1,755 subjects were grouped in the present example. In a training set and a testing set, 422 healthy persons, 433 patients with chronic liver disease (CLD) confirmed by liver puncture biopsy and 900 patients with HCC confirmed by liver histopathology were subjected to fasting for 12 h and then detected by ultrahigh performance liquid chromatography to obtain contents of metabolites including bile acids, fatty acids, organic acids, carbohydrates and amino acids and the like in serum/plasma samples and corresponding clinical indicators. In the present invention, test samples were approved by the Local Ethics Committee and informed consent of all the subjects.

### (1) Collection and Preparation of Serum Samples

5 mL of fasting venous blood was collected and placed in a plastic centrifuge tube.

### Preparation of Serum:

1) A serum preparation tube was slowly rotated for 5 times.
2) The test tube was vertically placed on a test tube rack at room temperature (about 25° C.) for 1.5 h.
3) The test tube was centrifuged at 2,500 rpm for 10 min (4° C.).
4) A supernatant (about 2.5 mL) was sucked into plastic centrifuge tubes (eppendorf, 1.5 mL centrifuge tube) by a pipette, and 0.5 mL of serum was frozen in each tube.
5) The centrifuge tubes were marked with sample numbers.
6) The centrifuge tubes were rapidly placed in a refrigerator at −80° C.

### (2) Detection of Clinical Indicators in Serum

Hematological and biochemical tests were carried out by using an LH750 hematology analyzer and a Synchron DXC800 clinical system (Beckman Coulter, the United States of America) according to test schemes of manufacturers. Hyaluronic acid and laminin in blood were detected by using a chemiluminescence immunoanalyzer (LUMO, Shinova Systems, Shanghai, China). Coagulation functions were detected by using a coagulation function measuring instrument (STAGO Compact, Diagnostica Stago, France). A blood HBV-DNA test was carried out by using a real-time polymerase chain reaction system (LightCycler 480, Roche, the United States of America).

### (3) Detection of Cholic Acids in Serum Samples

Preparation of samples: 100 μL of serum was added into a 1.5 mL centrifuge tube, and then 150 μL of methanol (containing an internal standard, 50 nM deuterated-CA (cholic acid), deuterated-UDCA (ursodeoxycholic acid) and deuterated-LCA (lithocholic acid)) was added. A mixed solution was subjected to vortex oscillation for uniform mixing for 10 min, standing for 10 min, and centrifugation at 13,500 rpm at 4° C. for 20 min, and a supernatant was taken for analysis by UPLC-TQMS (ultrahigh performance liquid chromatography-triple quadrupole mass spectrometry).

Test with analytical instruments: UPLC-TQMS: A Waters ultrahigh performance liquid chromatography system (Waters, the United States of America) equipped with a binary solvent controller and a sample control chamber was used. A Waters XEVO triple quadrupole mass spectrometer (Waters, the United States of America) equipped with a dual-electrospray ion source was used.

Conditions for chromatography: A UPLC BEH C18 chromatographic column (100 mm×2.1 mm, 1.7 μm) at a temperature of 45° C. was used. A mobile phase A including water (containing 0.1% of formic acid) and a mobile phase B including acetonitrile (containing 0.1% of formic acid) were used. A flow rate of 0.4 mL/min and an injection volume of 5 μL were adopted. Conditions for gradient elution were as follows: 0-1 min (5% B), 1-5 min (5-25% B), 5-15.5 min (25-40% B), 15.5-17.5 min (40-95% B), 17.5-19 min (95% B), 19-19.5 min (95-5% B), 19.6-21 min (5% B).

Conditions for mass spectrometry: An electrospray ion source was used in a negative ion scanning mode (ESI−) under the following specific conditions: voltage of a capillary tube, 1.2 kV; voltage of a cone hole, 55 V; voltage of an extraction cone hole, 4 V; temperature of an ion source, 150° C.; temperature of a solvent removal gas, 550° C.; flow rate of a reverse cone hole, 50 L/h; flow rate of a solvent removal gas, 650 L/h; resolution of a low mass area, 4.7; resolution of a high mass area, 15; and a multi-response detection mode for collecting data.

### (4) Detection of Free Fatty Acids in Serum Samples

Preparation of samples: 30 μL of serum was taken, 500 μL of isopropanol/n-hexane/2M phosphoric acid (40:10:1) and 10 μL of an isotope-labeled C19:0-d37 internal standard solution (5 μg/mL) were added, and a mixed solution was subjected to vortex treatment for 2 min, followed by standing at room temperature for 20 min. 400 μL of n-hexane and 300 μL of water were added, vortex treatment was performed for 2 min, centrifugation was performed at 12,000 rpm for 5 min, and then 400 μL of a supernatant was taken. 400 μL of n-hexane was added into the remaining solution, vortex treatment was performed for 2 min. centrifugation was performed at 12,000 rpm for 5 min, and then 400 μL of a supernatant was taken. The supernatants were combined, followed by vacuum drying at room temperature. 80 μL of methanol was added into a dried centrifuge tube for re-dissolution and then analyzed.

Test with analytical instruments: UPLC-TQMS: A Waters ultrahigh performance liquid chromatography system (Waters, the United States of America) equipped with a binary solvent controller and a sample control chamber was used. A Waters XEVO triple quadrupole mass spectrometer (Waters, the United States of America) equipped with a dual-electrospray ion source was used.

Conditions for chromatography: A UPLC BEH C18 chromatographic column (100 mm×2.1 mm, 1.7 μm) at a temperature of 40° C. was used. A mobile phase A including water and a mobile phase B including acetonitrile and isopropanol at a volume ratio of 8:2 were used. A flow rate of 0.4 mL/min and an injection volume of 5 μL were adopted. Conditions for gradient elution were as follows: 0-2 min: 70% B. 2-5 min: 70%-75% B. 5-10 min: 75%-80% B. 10-13 min: 80%-90% B, 13-16 min: 90%-100% B, 16-21 min: 100% B. 21-22.5 min: 100%-70% B, 22.5-24 min: 70% B. The total analysis time was 24 min.

Conditions for mass spectrometry: An electrospray ion source was used in a negative ion scanning mode (ESI−) under the following specific conditions: voltage of a capillary tube, 2.5 kV; voltage of a cone hole, 55 V; voltage of an extraction cone hole, 4 V; temperature of an ion source, 120° C.; temperature of a solvent removal gas, 450° C.; flow rate of a reverse cone hole, 50 L/h; flow rate of a solvent removal gas, 650 L/h; resolution of a low mass area, 4.7; resolution of a high mass area, 15; voltage of a detector, 2,390 V; scanning time, 0.35 s; scanning time interval, 0.02 s; and mass-charge ratio range, m/z 50-1,000. The locked mass number was 554.2615.

### (5) Detection of Amino Acids in Serum Samples

Preparation of samples: 40 μL of serum was taken, 500 μL of a mixed solvent of methanol and acetonitrile (1:9, v:v) was added, and vortex oscillation was performed for 2 min. A mixed solution was placed in a centrifuge tube at −20° C. for 10 min to promote protein precipitation, and centrifugation was performed at 12,000 rpm at 4° C. for 15 min. 20 μL of a supernatant was taken, followed by vacuum drying at room temperature. 100 μL of a mixed solvent of methanol and water (1:1, v:v, containing 1 μg/mL of dichlorophenylalanine as an internal standard) was added into a dried centrifuge tube for re-dissolution and then analyzed.

Test with analytical instruments: UPLC-TQMS: A Waters ultrahigh performance liquid chromatography system (Waters, the United States of America) equipped with a binary solvent controller and a sample control chamber was used. A Waters XEVO triple quadrupole mass spectrometer (Waters, the United States of America) equipped with a dual-electrospray ion source was used.

Conditions for chromatography: A UPLC BEH C18 chromatographic column (100 mm×2.1 mm, 1.7 μm) at a temperature of 40° C. was used. A mobile phase A including water (containing 0.1% of formic acid) and a mobile phase B including acetonitrile (containing 0.1% of formic acid) were used. A flow rate of 0.4 mL/min and an injection volume of 5 μL were adopted. Conditions for gradient elution were as follows: 0-0.5 min (1% B), 0.5-9 min (1-20% B), 9-11 min (20-75% B), 11-16 min (75-99% B), 16-16.5 min (99% B).

Conditions for mass spectrometry: An electrospray ion source was used in a negative ion scanning mode (ESI−) under the following specific conditions: voltage of a capillary tube, 3.0; voltage of a cone hole, 55 V; voltage of an extraction cone hole, 4 V; temperature of an ion source, 150° C.; temperature of a solvent removal gas, 450° C.; flow rate of a reverse cone hole, 50 L/h; flow rate of a solvent removal gas, 800 L/h; resolution of a low mass area, 4.7; resolution of a high mass area, 15; and a multi-response detection mode for collecting data.

### (6) Detection of Triglycerides in Serum Samples

Triglycerides in serum were detected by an enzyme colorimetric method.

### (7) Liver Biopsy

All patients were subjected to ultrasound-guided liver puncture biopsy. By means of a “7-point” baseline sampling method, samples were collected at junctions of carcinoma tissues and adjacent hepatic tissues at 1:1 at 12, 3, 6 and 9 point positions of a tumor. At least one sample was collected in the tumor. One hepatic tissue was collected at a distance equal to or less than 1 cm (adjacent carcinoma side) and at a distance greater than 1 cm (distant carcinoma side) separately. The above samples were fixed with 10% formalin for 12-24 h and embedded in paraffin, and tissue sections were stained with hematoxylin-eosin and Masson. During pathological evaluation, the samples were separately evaluated by three pathologists from Shanghai Medical College of Fudan University based on a blind method, and the consistency of results was validated by a Kappa test. When the evaluation results were failed in the Kappa test, the samples were reanalyzed to obtain consistent results.

### (8) Bioinformatics Method

In the present invention, 422 healthy persons, 433 patients with CLD and 900 patients with HCC were randomly divided into a training set and a testing set at a ratio of 70%:30%. In the training set, in order to distinguish the healthy persons and the patients with HCC and distinguish the patients with CLD and the patients with HCC, candidate biomarkers were selected and identified by using a single-factor Wilcoxon rank sum test and LASSO, a random forest model was used for evaluating candidate variables and building models, and then the models were validated in the testing set and an independent validation set, respectively.

Through the above research, it is found that levels of a set of biomarkers including one or more of taurocholic acid, taurochenodeoxycholic acid, glycocholic acid, glycochenodeoxycholic acid, trans-linoleic acid (C18:2n6t), maltotriose, maltose and/or lactose, α-linolenic acid, β-alanine, sebacic acid, 2-methylvaleric acid, valeric acid, isovaleric acid and caproic acid have predictive and diagnostic capabilities; and the ratio of a secondary bile acid to a primary bile acid and/or the ratio of a combination of glycine and a primary bile acid to a combination of taurine and a primary bile acid also have predictive and diagnostic capabilities. The primary bile acid is selected from cholic acid and chenodeoxycholic acid. The secondary bile acid includes deoxycholic acid, lithocholic acid and ursodeoxycholic acid.

### Example 2 Quantitative Detection of the Concentration of Diagnostic Markers

Standard curves were drawn according to ratios of concentrations of standard solutions of diagnostic markers to be tested to areas of the corresponding diagnostic markers to be tested and stable isotope internal standards equivalent to the diagnostic markers to be tested, and isotope internal standards were used for quantitative determination. Meanwhile, isotope internal standards were added into samples to carry out quality control in a sample detection process.

Detection methods were referred to those in Example 1.

### Example 3 Distinguishing Between Healthy Controls and HCC

In a training set, 630 patients with HCC and 296 healthy persons were involved. In a testing set, 270 patients with HCC and 126 healthy persons were involved. By means of the biomarkers obtained in Example 1, the possibility of HCC in the above subjects was outputted by a random forest model of biomarker combinations trained in the training set. In addition, optimal cutoff values were found based on Youden optimal values in receiver operating characteristic (ROC) analysis, so as to evaluate the overall capability of the model to distinguish the patients with HCC and the healthy persons. The testing set was tested. Results are shown in Table 1 and Table 2.

TABLE 1
Area under an ROC curve and a confidence interval

| | | Asymptotic 95% | |
|---|---|---|---|
| | | confidence interval | |
| | Area under an | Lower | Upper |
| Marker | ROC curve | limit | limit |
| β-alanine | 0.991638 | 0.988038 | 0.995237 |
| Sebacic acid | 0.985816 | 0.981036 | 0.990597 |
| 2-methylvaleric acid | 0.777396 | 0.747347 | 0.807444 |
| Valeric acid | 0.743023 | 0.709788 | 0.776259 |
| Isovaleric acid | 0.871707 | 0.85051 | 0.892905 |
| Caproic acid | 0.889894 | 0.870825 | 0.908963 |
| Maltotriose | 0.968646 | 0.961271 | 0.976021 |
| Maltose/lactose | 0.953875 | 0.945164 | 0.962585 |
| α-linolenic acid | 0.878677 | 0.861537 | 0.895817 |
| Taurochenodeoxycholic acid | 0.886618 | 0.869509 | 0.903727 |
| Glycocholic acid | 0.826936 | 0.806288 | 0.847584 |
| Taurocholic acid | 0.815048 | 0.793956 | 0.83614 |
| Glycochenodeoxycholic acid | 0.756166 | 0.731345 | 0.770987 |
| Glycochenodeoxycholic | 0.860106 | 0.839755 | 0.880457 |
| acid/taurochenodeoxycholic acid | | | |
| Deoxycholic acid/cholic acid | 0.800368 | 0.770117 | 0.830619 |
| Lithocholic acid/chenodeoxycholic acid | 0.87607 | 0.847559 | 0.894581 |
| Ursodeoxycholic acid/chenodeoxycholic acid | 0.777328 | 0.748165 | 0.80649 |
| Linoleic acid | 0.735545 | 0.706502 | 0.764588 |
| Alpha-fetoprotein | 0.707 | 0.658 | 0.712 |
| β-alanine + alpha-fetoprotein | 0.999168 | 0.999679 | 0.998657 |
| Sebacic acid + alpha-fetoprotein | 0.996377 | 0.993798 | 0.998956 |
| 2-methylvaleric acid + alpha-fetoprotein | 0.975185 | 0.969761 | 0.980609 |
| Valeric acid + alpha-fetoprotein | 0.9674 | 0.960891 | 0.973909 |
| Isovaleric acid + alpha-fetoprotein | 0.982526 | 0.978254 | 0.986799 |
| Caproic acid + alpha-fetoprotein | 0.987285 | 0.98388 | 0.99069 |
| Maltotriose + alpha-fetoprotein | 0.995584 | 0.993222 | 0.997946 |
| Maltose/lactose + alpha-fetoprotein | 0.995975 | 0.994061 | 0.99789 |
| α-linolenic acid + alpha-fetoprotein | 0.986149 | 0.98231 | 0.989988 |
| Taurochenodeoxycholic acid + alpha-fetoprotein | 0.986183 | 0.982302 | 0.990065 |
| Glycocholic acid + alpha-fetoprotein | 0.97247 | 0.965923 | 0.979017 |
| Taurocholic acid + alpha-fetoprotein | 0.969708 | 0.962522 | 0.976895 |
| Glycochenodeoxycholic acid + alpha-fetoprotein | 0.961419 | 0.953277 | 0.969562 |
| Glycochenodeoxycholic acid/ | 0.981219 | 0.976169 | 0.986269 |
| taurochenodeoxycholic acid + alpha-fetoprotein | | | |
| Deoxycholic acid/cholic acid + alpha-fetoprotein | 0.953052 | 0.944293 | 0.961812 |
| Lithocholic acid/chenodeoxycholic | 0.957713 | 0.949482 | 0.965944 |
| acid + alpha-fetoprotein | | | |
| Ursodeoxycholic acid/chenodeoxycholic | 0.947598 | 0.937885 | 0.957311 |
| acid + alpha-fetoprotein | | | |
| Linoleic acid + alpha-fetoprotein | 0.943999 | 0.934087 | 0.953912 |
| Isovaleric acid + caproic acid + maltotriose + α- | 0.995383 | 0.993655 | 0.997111 |
| linolenic acid | | | |
| Isovaleric acid + caproic acid + maltotriose + α- | 0.999457 | 0.999823 | 0.99909 |
| linolenic acid + alpha-fetoprotein | | | |

TABLE 2

| Marker | Cutoff value | Sensitivity | Specificity |
|---|---|---|---|
| β-alanine | 33.92 | 0.96 | 0.97 |
| Sebacic acid | 0.19 | 0.99 | 0.94 |
| 2-methylvaleric acid | 0.2 | 0.7 | 1 |
| Valeric acid | 0.68 | 0.76 | 0.98 |
| Isovaleric acid | 0.42 | 0.73 | 0.85 |
| Caproic acid | 0.42 | 0.7 | 0.93 |
| Maltotriose | 0.12 | 0.93 | 0.96 |
| Maltose/lactose | 3.08 | 0.85 | 0.99 |
| α-linolenic acid | 3.08 | 0.85 | 0.99 |
| Taurochenodeoxycholic acid | 32.5 | 0.81 | 0.79 |
| Glycocholic acid | 0.14 | 0.8 | 0.83 |
| Taurocholic acid | 0.52 | 0.77 | 0.74 |
| Glycochenodeoxycholic acid | 0.17 | 0.76 | 0.8 |
| Glycochenodeoxycholic | 2.08 | 0.85 | 0.82 |
| acid/taurochenodeoxycholic acid | | | |
| Deoxycholic acid/cholic acid | 9.33 | 0.81 | 0.79 |
| Lithocholic acid/chenodeoxycholic acid | 2.73 | 0.73 | 0.83 |
| Ursodeoxycholic acid/chenodeoxycholic acid | 0.07 | 0.74 | 0.76 |
| Linoleic acid | 0.31 | 0.7 | 0.77 |
| Alpha-fetoprotein | 0.35 | 0.58 | 0.66 |
| β-alanine + alpha-fetoprotein | 0.66 | 0.99 | 0.99 |
| Sebacic acid + alpha-fetoprotein | 0.72 | 0.99 | 1 |
| 2-methylvaleric acid + alpha-fetoprotein | 0.64 | 0.88 | 1 |
| Valeric acid + alpha-fetoprotein | 0.6 | 0.88 | 1 |
| Isovaleric acid + alpha-fetoprotein | 0.63 | 0.92 | 0.96 |
| Caproic acid + alpha-fetoprotein | 0.73 | 0.91 | 0.99 |
| Maltotriose + alpha-fetoprotein | 0.11 | 0.99 | 0.99 |
| Maltose/lactose + alpha-fetoprotein | 0.41 | 0.98 | 1 |
| α-linolenic acid + alpha-fetoprotein | 0.8 | 0.92 | 0.99 |
| Taurochenodeoxycholic acid + alpha- | 0.64 | 0.93 | 0.98 |
| fetoprotein | | | |
| Glycocholic acid + alpha-fetoprotein | 0.65 | 0.91 | 0.99 |
| Taurocholic acid + alpha-fetoprotein | 0.74 | 0.89 | 0.99 |
| Glycochenodeoxycholic acid + alpha-fetoprotein | 0.53 | 0.91 | 0.98 |
| Glycochenodeoxycholic acid/ | 0.56 | 0.93 | 0.96 |
| taurochenodeoxycholic acid + alpha-fetoprotein | | | |
| Deoxycholic acid/cholic acid + alpha-fetoprotein | 0.33 | 0.88 | 1 |
| Lithocholic acid/chenodeoxycholic | 0.73 | 0.88 | 1 |
| acid + alpha-fetoprotein | | | |
| Ursodeoxycholic acid/chenodeoxycholic | 0.55 | 0.88 | 1 |
| acid + alpha-fetoprotein | | | |
| Linoleic acid + alpha-fetoprotein | 0.32 | 0.88 | 1 |
| Isovaleric acid + caproic acid + maltotriose + | 0.91 | 0.93 | 1 |
| α-linolenic acid | | | |
| Isovaleric acid + caproic acid + maltotriose + | 0.91 | 0.99 | 1 |
| α-linolenic acid + alpha-fetoprotein | | | |

### Example 4 Distinguishing Between Liver Cirrhosis and HCC

In a training set, 630 patients with HCC and 303 patients with liver cirrhosis were involved. In a testing set, 270 patients with HCC and 130 patients with liver cirrhosis were involved. By means of the biomarkers obtained in Example 1, the possibility of HCC in the above subjects was outputted by a random forest model of biomarker combinations trained in the training set. In addition, optimal cutoff values were found based on Youden optimal values in ROC analysis, so as to evaluate the overall capability of the model to distinguish the patients with HCC and the patients with liver cirrhosis. The testing set was tested. Results are shown in Table 3 and Table 4.

TABLE 3

| | | Asymptotic 95% | |
|---|---|---|---|
| | | confidence interval | |
| | Area under an | Lower | Upper |
| Marker | ROC curve | limit | limit |
| β-alanine | 0.766529 | 0.712166 | 0.820892 |
| Sebacic acid | 0.737715 | 0.688812 | 0.786618 |
| 2-methylvaleric acid | 0.785012 | 0.735032 | 0.834993 |
| Valeric acid | 0.808047 | 0.760881 | 0.855213 |
| Isovaleric acid | 0.84694 | 0.812912 | 0.880968 |
| Caproic acid | 0.841384 | 0.80206 | 0.880708 |
| Maltotriose | 0.958622 | 0.968289 | 0.948955 |
| Maltose/lactose | 0.939217 | 0.951825 | 0.926609 |
| α-linolenic acid | 0.907499 | 0.930909 | 0.88409 |
| Taurochenodeoxycholic acid | 0.835437 | 0.797419 | 0.873454 |
| Glycocholic acid | 0.83418 | 0.796048 | 0.872312 |
| Taurocholic acid | 0.848085 | 0.812975 | 0.883195 |
| Glycochenodeoxycholic acid | 0.831835 | 0.799931 | 0.863739 |
| Glycochenodeoxycholic | 0.622459 | 0.672803 | 0.572115 |
| acid/taurochenodeoxycholic acid | | | |
| Deoxycholic acid/cholic acid | 0.572537 | 0.624467 | 0.520608 |
| Lithocholic acid/chenodeoxycholic acid | 0.794338 | 0.831269 | 0.757407 |
| Ursodeoxycholic acid/chenodeoxycholic acid | 0.695471 | 0.744478 | 0.646465 |
| Linoleic acid | 0.641529 | 0.684463 | 0.598595 |
| Alpha-fetoprotein | 0.663 | 0.643 | 0.699 |
| β-alanine + alpha-fetoprotein | 0.8597 | 0.8925 | 0.826899 |
| Sebacic acid + alpha-fetoprotein | 0.841551 | 0.871088 | 0.812015 |
| 2-methylvaleric acid + alpha-fetoprotein | 0.883404 | 0.912243 | 0.854566 |
| Valeric acid + alpha-fetoprotein | 0.904903 | 0.928534 | 0.881272 |
| Isovaleric acid + alpha-fetoprotein | 0.917104 | 0.937122 | 0.897086 |
| Caproic acid + alpha-fetoprotein | 0.918444 | 0.940051 | 0.896837 |
| Maltotriose + alpha-fetoprotein | 0.974229 | 0.981291 | 0.967168 |
| Maltose/lactose + alpha-fetoprotein | 0.965909 | 0.974371 | 0.957447 |
| α-linolenic acid + alpha-fetoprotein | 0.945751 | 0.96237 | 0.929131 |
| Taurochenodeoxycholic acid + alpha-fetoprotein | 0.898006 | 0.921893 | 0.87412 |
| Glycocholic acid + alpha-fetoprotein | 0.888234 | 0.914304 | 0.862165 |
| Taurocholic acid + alpha-fetoprotein | 0.894488 | 0.92033 | 0.868647 |
| Glycochenodeoxycholic acid + alpha-fetoprotein | 0.895326 | 0.91835 | 0.872302 |
| Glycochenodeoxycholic acid/ | 0.719539 | 0.749799 | 0.689279 |
| taurochenodeoxycholic acid + alpha-fetoprotein | | | |
| Deoxycholic acid/cholic acid + alpha-fetoprotein | 0.759298 | 0.790725 | 0.72787 |
| Lithocholic acid/chenodeoxycholic | 0.86788 | 0.893501 | 0.842259 |
| acid + alpha-fetoprotein | | | |
| Ursodeoxycholic acid/chenodeoxycholic | 0.710576 | 0.740859 | 0.680294 |
| acid + alpha-fetoprotein | | | |
| Linoleic acid + alpha-fetoprotein | 0.79861 | 0.827427 | 0.769792 |
| Isovaleric acid + caproic acid + maltotriose + α- | 0.994946 | 0.997361 | 0.992531 |
| linolenic acid | | | |
| Isovaleric acid + caproic acid + maltotriose + α- | 0.997906 | 0.999154 | 0.996658 |
| linolenic acid + alpha-fetoprotein | | | |

TABLE 4

| Marker | Cutoff value | Sensitivity | Specificity |
|---|---|---|---|
| β-alanine | 30.49 | 0.71 | 0.92 |
| Sebacic acid | 0.1058675 | 0.776 | 0.717 |
| 2-methylvaleric acid | 0.1951372 | 0.608 | 0.986 |
| Valeric acid | 0.4065446 | 0.689 | 0.899 |
| Isovaleric acid | 0.3495954 | 0.784 | 0.764 |
| Caproic acid | 0.3758446 | 0.689 | 0.884 |
| Maltotriose | 0.2698011 | 0.847 | 0.986 |
| Maltose/lactose | 9.1666464 | 0.775 | 1 |
| α-linolenic acid | 28.518139 | 0.876 | 0.811 |
| Taurochenodeoxycholic acid | 2.3307873 | 0.716 | 0.864 |
| Glycocholic acid | 6.015907 | 0.716 | 0.837 |
| Taurocholic acid | 1.5681643 | 0.73 | 0.833 |
| Glycochenodeoxycholic acid | 6.9948895 | 0.757 | 0.762 |
| Glycochenodeoxycholic | 3.5920052 | 0.781 | 0.459 |
| acid/taurochenodeoxycholic acid | | | |
| Deoxycholic acid/cholic acid | 1.7027639 | 0.777 | 0.419 |
| Lithocholic acid/chenodeoxycholic acid | 0.069606 | 0.726 | 0.851 |
| Ursodeoxycholic acid/chenodeoxycholic acid | 0.2087658 | 0.759 | 0.703 |
| Linoleic acid | 6.0178277 | 0.75 | 0.789 |
| Alpha-fetoprotein | 0.35 | 0.45 | 0.609 |
| β-alanine + alpha-fetoprotein | 0.835477 | 0.847 | 0.703 |
| Sebacic acid + alpha-fetoprotein | 0.8876754 | 0.758 | 0.77 |
| 2-methylvaleric acid + alpha-fetoprotein | 0.7690559 | 0.99 | 0.622 |
| Valeric acid + alpha-fetoprotein | 0.8786469 | 0.911 | 0.73 |
| Isovaleric acid + alpha-fetoprotein | 0.9302644 | 0.731 | 0.946 |
| Caproic acid + alpha-fetoprotein | 0.9554354 | 0.692 | 0.946 |
| Maltotriose + alpha-fetoprotein | 0.8750513 | 0.909 | 0.986 |
| Maltose/lactose + alpha-fetoprotein | 0.9522742 | 0.87 | 0.986 |
| α-linolenic acid + alpha-fetoprotein | 0.8345525 | 0.89 | 0.892 |
| Taurochenodeoxycholic acid + alpha-fetoprotein | 0.8741445 | 0.868 | 0.77 |
| Glycocholic acid + alpha-fetoprotein | 0.8951366 | 0.843 | 0.743 |
| Taurocholic acid + alpha-fetoprotein | 0.8788622 | 0.878 | 0.757 |
| Glycochenodeoxycholic acid + alpha-fetoprotein | 0.9039795 | 0.798 | 0.878 |
| Glycochenodeoxycholic acid/ | 0.8872876 | 0.802 | 0.946 |
| taurochenodeoxycholic acid + alpha-fetoprotein | | | |
| Deoxycholic acid/cholic acid + alpha-fetoprotein | 0.9017668 | 0.806 | 0.946 |
| Lithocholic acid/chenodeoxycholic | 0.8619665 | 0.733 | 0.878 |
| acid + alpha-fetoprotein | | | |
| Ursodeoxycholic acid/chenodeoxycholic | 0.8883676 | 0.89 | 0.946 |
| acid + alpha-fetoprotein | | | |
| Linoleic acid + alpha-fetoprotein | 0.9187449 | 0.758 | 0.946 |
| Isovaleric acid + caproic | 0.8211712 | 0.969 | 0.986 |
| acid + maltotriose + α-linolenic acid | | | |
| Isovaleric acid + caproic | 0.9752508 | 0.967 | 1 |
| acid + maltotriose + α-linolenic acid + alpha- | | | |
| fetoprotein | | | |

### Example 5 Distinguishing Between a Healthy Group and an HCC Group by Using a Biomarker Combination of Taurocholic Acid, Taurochenodeoxycholic Acid, Glycocholic Acid and Trans-Linoleic Acid (C18:2n6t)

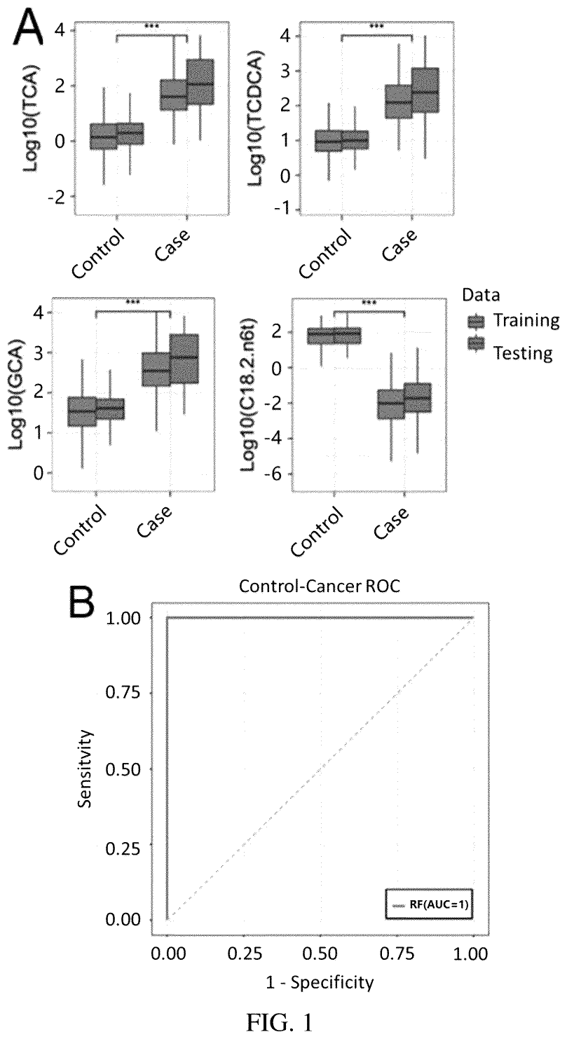

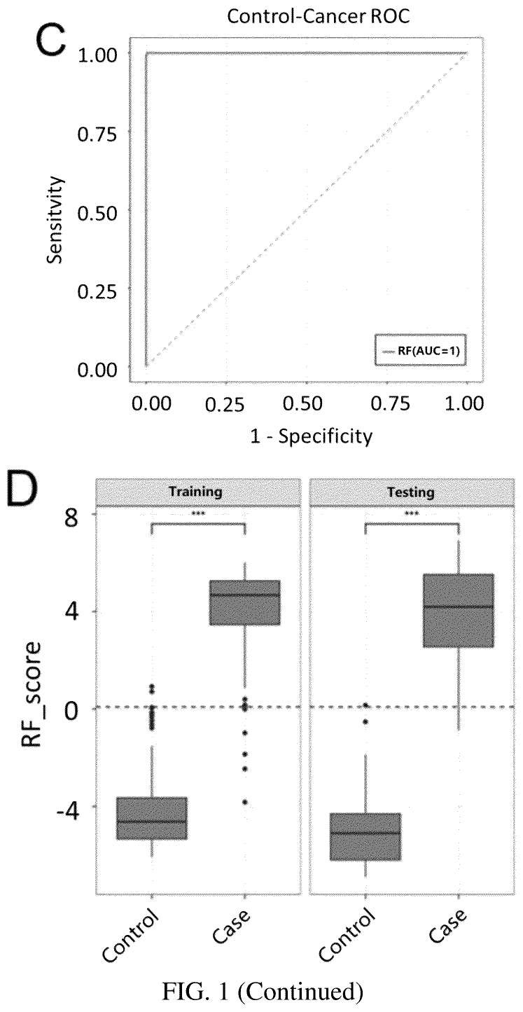

In a training set, 630 patients with HCC and 296 healthy persons were involved. In a testing set, 270 patients with HCC and 126 healthy persons were involved. The possibility of HCC in the above subjects was outputted by a random forest model of a biomarker combination trained in the training set. In addition, optimal cutoff values were found based on Youden optimal values in ROC analysis, so as to evaluate the overall capability of the model to distinguish the patients with HCC and the healthy persons. Results are shown in FIG. 1 and Table 5. In the training set, the area under an ROC curve and the 95% confidence interval are 1.000 (95% CI 0.999-1.000), the optimal cutoff value is 0.078, and the sensitivity and the specificity at the optimal cutoff value are 99.7% and 100%, respectively. In the testing set, the area under an ROC curve and the 95% confidence interval are 1.000 (95% CI 0.999-1.000), the optimal cutoff value is 0.078, and the sensitivity and the specificity at the optimal cutoff value are 99.2% and 100%, respectively. Levels of markers were measured for the subjects, and then the measured values were introduced into the random forest model. When a score threshold of an individual is greater than 0.078, it is indicated that the individual has a higher risk of HCC. When a score threshold of an individual is less than 0.078, it is indicated that the individual has a lower risk of HCC.

TABLE 5
Diagnostic capability of a biomarker combination

| Model | | | Area under an | | | Cutoff |
|---|---|---|---|---|---|---|
| method | Group | Data set | ROC curve | Specificity | Sensitivity | value |
| Random | Control- | Training | 0.997 | 99.15% | 98.55% | 0.08 |
| forest model | HCC | set | (0.992-1) | | | |
| FIB-4 index | Control- | Training | 0.839 | 82.10% | 69.41% | 0.48 |
| | HCC | set | (0.811-0.866) | | | |
| APRI index | Control- | Training | 0.975 | 91.98% | 93.24% | 0.36 |
| | HCC | set | (0.965-0.984) | | | |
| AST/ALT | Control- | Training | 0.738 | 63.27% | 73.31% | 0.52 |
| ratio | HCC | set | (0.702-0.774) | | | |
| Random | Control- | Validation | 1 | 99.25% | 97.24% | 0.08 |
| forest model | HCC | set | (0.999-1) | | | |
| FIB-4 index | Control- | Validation | 0.895 | 85.83% | 77.46% | 0.48 |
| | HCC | set | (0.855-0.93) | | | |
| APRI index | Control- | Validation | 0.988 | 93.70% | 95.77% | 0.36 |
| | HCC | set | (0.975-0.996) | | | |
| AST/ALT | Control- | Validation | 0.501 | 55.12% | 47.18% | 0.52 |
| ratio | HCC | set | (0.43-0.569) | | | |

### Example 6 Distinguishing Between a CLD Group and an HCC Group by Using a Biomarker Combination of Taurocholic Acid, Taurochenodeoxycholic Acid, Glycocholic Acid and Trans-Linoleic Acid (C18:2n6t)

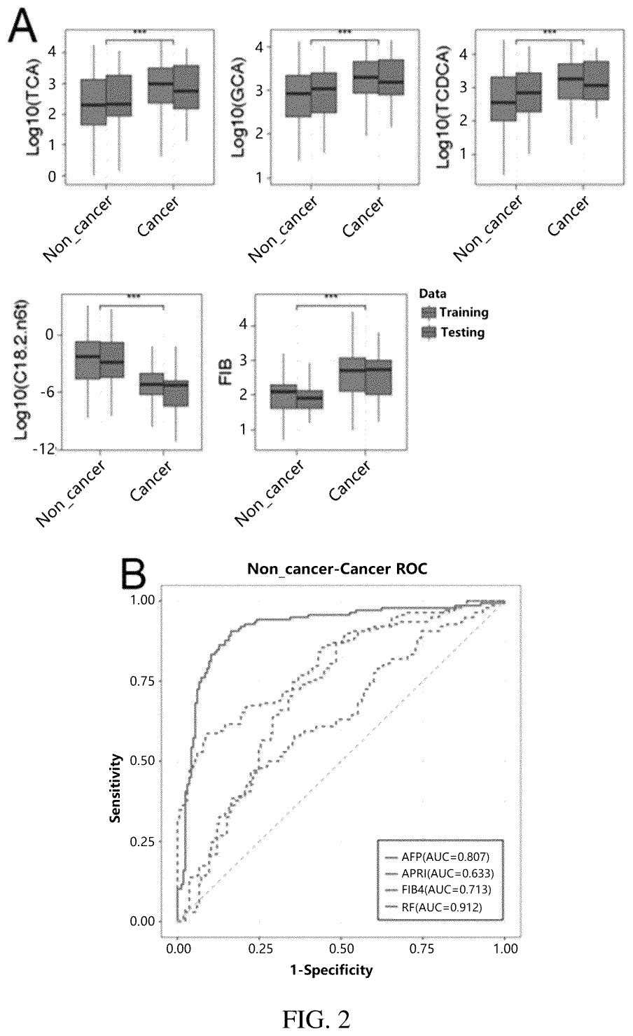

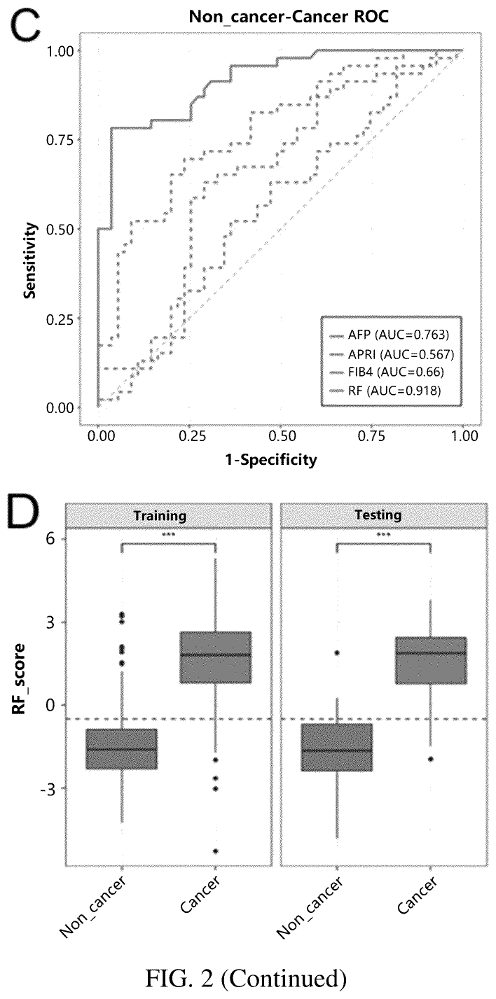

In a training set, 630 patients with HCC and 303 patients with CLD were involved. In a testing set, 303 patients with HCC and 130 patients with CLD were involved. The possibility of HCC in the above subjects was outputted by a random forest model of a biomarker combination trained in the training set. In addition, optimal cutoff values were found based on Youden optimal values in ROC analysis, so as to evaluate the overall capability of the model to distinguish the patients with HCC and the patients with CLD. Results are shown in FIG. 2 and Table 6. In the training set, the area under an ROC curve and the 95% confidence interval are 0.912 (95% CI 0.874-0.946), the optimal cutoff value is −0.502, and the sensitivity and the specificity at the optimal cutoff value are 83.6% and 90.6%, respectively. In the testing set, the area under an ROC curve and the 95% confidence interval are 0.918, the optimal cutoff value is −0.502, and the sensitivity and the specificity at the optimal cutoff value are 81.8% and 80.4%, respectively. Levels of markers were measured for the subjects, and then the measured values were introduced into the random forest model. When a score threshold of an individual is greater than −0.502, it is indicated that the individual has a higher risk of HCC. When a score threshold of an individual is less than −0.502, it is indicated that the individual has a lower risk of HCC.

TABLE 6
Diagnostic capability of a biomarker combination

| Model | | | Area under an | | | Cutoff |
|---|---|---|---|---|---|---|
| method | Group | Data set | ROC curve | Specificity | Sensitivity | value |
| Random | Healthy control- | Training | 0.912 | 83.6% | 90.6% | −0.50 |
| forest | hepatocellular | set | (0.874-0.946) | | | |
| model | carcinoma | | | | | |
| FIB-4 | Healthy control- | Training | 0.713 | 51.5% | 87.0% | 0.45 |
| | hepatocellular | set | (0.655-0.769) | | | |
| | carcinoma | | | | | |
| APRI | Healthy control- | Training | 0.633 | 77.0% | 47.1% | 0.44 |
| index | hepatocellular | set | (0.57-0.699) | | | |
| | carcinoma | | | | | |
| AST/ALT | Healthy control- | Training | 0.367 | 53.3% | 76.1% | 0.46 |
| ratio | hepatocellular | set | (0.306-0.427) | | | |
| | carcinoma | | | | | |
| Alpha- | Healthy control- | Training | 0.707 | 66.0% | 58.7% | 0.35 |
| fetoprotein | hepatocellular | set | (0.658-0.712) | | | |
| (AFP) | carcinoma | | | | | |
| Random | Liver cirrhosis- | Validation | 0.918 | 81.8% | 80.4% | −0.50 |
| forest | hepatocellular | set | (0.853-0.962) | | | |
| model | carcinoma | | | | | |
| FIB-4 | Liver cirrhosis- | Validation | 0.66 | 36.4% | 93.5% | 0.45 |
| | hepatocellular | set | (0.552-0.756) | | | |
| | carcinoma | | | | | |
| APRI | Liver cirrhosis- | Validation | 0.567 | 63.6% | 52.2% | 0.44 |
| index | hepatocellular | set | (0.451-0.676) | | | |
| | carcinoma | | | | | |
| AST/ALT | Liver cirrhosis- | Validation | 0.381 | 45.5% | 73.9% | 0.46 |
| ratio | hepatocellular | set | (0.277-0.499) | | | |
| | carcinoma | | | | | |
| Alpha- | Liver cirrhosis- | Validation | 0.663 | 60.9% | 45.7% | 0.35 |
| fetoprotein | hepatocellular | set | (0.643-0.699) | | | |
| | carcinoma | | | | | |

### Example 7

A method for diagnosis of hepatocellular carcinoma includes treating a biological sample of a subject according to the above examples and then subjecting a biomarker combination in the biological sample to quantitative detection by a chromatography-mass spectrometry method in combination with a metabolomics analysis method. The biomarker combination includes the effective biomarker combinations proven in the above examples. The chromatography-mass spectrometry method in combination with the metabolomics analysis method includes a liquid chromatography-mass spectrometry method in combination with the metabolomics analysis method and a gas chromatography-mass spectrometry method in combination with the metabolomics analysis method as used in the above examples.

1. A diagnostic device for hepatocellular carcinoma, wherein the device is used by determining levels of biomarkers in a biological sample of a subject as diagnostic indicators; the levels of biomarkers are selected from levels of one or more of taurocholic acid, taurochenodeoxycholic acid, glycocholic acid, glycochenodeoxycholic acid, trans-linoleic acid (C18:2n6t), maltotriose, maltose and/or lactose, α-linolenic acid, β-alanine, sebacic acid, 2-methylvaleric acid, valeric acid, isovaleric acid and caproic acid, and/or the ratio of a secondary bile acid to a primary bile acid, and/or the ratio of a combination of glycine and a primary bile acid to a combination of taurine and a primary bile acid; the primary bile acid is selected from cholic acid and chenodeoxycholic acid; and the secondary bile acid comprises deoxycholic acid, lithocholic acid and ursodeoxycholic acid.

2. The diagnostic device for hepatocellular carcinoma according to claim 1, wherein determination of the levels of biomarkers comprises the following steps: treating the biological sample of the subject and then subjecting a biomarker combination in the biological sample to quantitative detection by a chromatography-mass spectrometry method in combination with a metabolomics analysis method; and the chromatography-mass spectrometry method in combination with the metabolomics analysis method comprises a liquid chromatography-mass spectrometry method in combination with the metabolomics analysis method and a gas chromatography-mass spectrometry method in combination with the metabolomics analysis method.

3. The diagnostic device for hepatocellular carcinoma according to claim 1, wherein the diagnostic device is selected from a kit, a medical instrument, a computer system with a diagnostic module, and a detection device with a diagnostic module.

4. The diagnostic device for hepatocellular carcinoma according to claim 3, wherein the diagnostic module comprises an information acquisition module and a hepatocellular carcinoma diagnosis module; the information acquisition module is at least used for acquiring information of diagnostic indicators; and the hepatocellular carcinoma diagnostic module is at least used for performing the following operation: assessing whether a subject has hepatocellular carcinoma or liver cirrhosis based on the information of diagnostic indicators acquired by the information acquisition module.

5. The diagnostic device for hepatocellular carcinoma according to claim 3, wherein the kit comprises quantitative detection reagents for detecting diagnostic indicators.

6. The diagnostic device for hepatocellular carcinoma according to claim 1, wherein the diagnostic indicators comprise alpha-fetoprotein.

7. A biomarker combination for diagnosis of hepatocellular carcinoma, comprising one or more of taurocholic acid, taurochenodeoxycholic acid, glycocholic acid, glycochenodeoxycholic acid, trans-linoleic acid (C18:2n6t), maltotriose, maltose and/or lactose, α-linolenic acid, β-alanine, sebacic acid, 2-methylvaleric acid, valeric acid, isovaleric acid and caproic acid.

8. The biomarker combination according to claim 7, wherein the biomarker combination further comprises alpha-fetoprotein.

9. A method for diagnosis of hepatocellular carcinoma or liver cirrhosis, comprising quantitative detection of the biomarker combination according to claim 7.

10. A method for evaluating drugs for treatment of hepatocellular carcinoma or liver cirrhosis, comprising quantitative detection of the biomarker combination according to claim 7.

11. A method for diagnosis of hepatocellular carcinoma, comprising treating a biological sample of a subject and then subjecting a biomarker combination in the biological sample to quantitative detection by a chromatography-mass spectrometry method in combination with a metabolomics analysis method, wherein the biomarker combination comprises the biomarker combination according to claim 7; and the chromatography-mass spectrometry method in combination with the metabolomics analysis method comprises a liquid chromatography-mass spectrometry method in combination with the metabolomics analysis method and a gas chromatography-mass spectrometry method in combination with the metabolomics analysis method.

12. A method for diagnosis of hepatocellular carcinoma or liver cirrhosis, comprising quantitative detection of the biomarker combination according to claim 8.

13. A method for evaluating drugs for treatment of hepatocellular carcinoma or liver cirrhosis, comprising quantitative detection of the biomarker combination according to claim 8.

14. A method for diagnosis of hepatocellular carcinoma, comprising treating a biological sample of a subject and then subjecting a biomarker combination in the biological sample to quantitative detection by a chromatography-mass spectrometry method in combination with a metabolomics analysis method, wherein the biomarker combination comprises the biomarker combination according to claim 8; and the chromatography-mass spectrometry method in combination with the metabolomics analysis method comprises a liquid chromatography-mass spectrometry method in combination with the metabolomics analysis method and a gas chromatography-mass spectrometry method in combination with the metabolomics analysis method.
